(19) United States
(12) Patent Application Publication
(10) Pub. No.: US 2020/0246798 A1
(43) Pub. Date: Aug. 6, 2020

(54) MICROFLUIDIC DEVICE POSSESSING STRUCTURES ENABLING DIFFERENTIAL ANALYSIS OF A SINGLE CELL'S CONSTITUENTS

(71) Applicants: KONINKLIJKE PHILIPS N.V.; DANMARKS TEKNISKE UNIVERSITET

(72) Inventors: Pieter Jan VAN DER ZAAG, WAALRE (NL); Rodolphe Charly Willy MARIE, Kgs. Lyngby (DK); Dianne Arnoldina Margaretha Wilhelmina VAN STRIJP, `s-Hertogenbosch (NL); Tom OLESEN, Allerød (DK); Roland Cornelis Martinus VULDERS, EINDHOVEN (NL); Anders KRISTENSEN, Kgs. Lyngby (DK)

(21) Appl. No.: 15/776,221

(22) PCT Filed: Nov. 15, 2016
(86) PCT No.: PCT/EP2016/077621

§ 371 Date: May 15, 2018

(30) Foreign Application Priority Data
Nov. 20, 2015 (EP) 15195604.2

(51) Int. Cl. B01L 3/00; C12M 1/00

(52) U.S. Cl. CPC B01L 3/502761; C12M 47/06; B01L 2400/0622; B01L 2300/0816; B01L 2300/0864; B01L 2200/0668

(57) ABSTRACT

A method and a micro fluidic device comprising at least one micro fluidic structure for differential extraction of nuclear and extra-nuclear constituents of a single cell, said micro fluidic structure comprising a feeding channel for receiving a volume of a sample containing at least one cell, at least one trapping structure for capturing a single cell, and at least one output channel in fluid connection with the at least one trapping structure, wherein the at least one trapping structure extends from one side of the feeding channel substantially perpendicular to longitudinal axis of the feeding channel, the at least one trapping structure possessing an aperture at its end opposite to the fluid channel and in fluid communication with an output channel, said aperture being configured to provide a narrow section such that the nucleus of a cell captured in the trapping structure cannot pass through said narrow section into the output channel.

### FIELD OF THE INVENTION

The invention relates to microfluidic devices for capturing and subsequently analyzing and/or characterizing single cells.

### BACKGROUND OF THE INVENTION

The technology of nucleic acid sequencing rapidly developed to the level that sequencing is applied in the diagnosis of cancer. Typically, mutations in the DNA of a cancer patient are determined for assessing which type of treatment the patient has to undergo and which type of drug is to be administered. Indeed, some cases exist in which a direct link between mutations in the DNA of a cancerous tissue and the drug to be used for treating this type of cancer could have been established. For example, the HER2-neu gene leads to an overexpression of the HER2 receptor which stimulates cell division. In such cancers, administration of Herceptin offers an effective treatment. However, it is very difficult establishing a direct link between cancer type, DNA mutations, and effective drug, because the DNA of tumor cells contain many mutations and it is difficult to assess which mutation drives the cancer, and which mutations are passerby mutations.

Recently, a more rewarding approach of assessing tumors by nucleic acid sequencing and assessing what therapy should be applied has been established (Verhaegh, W. et al. (2014): Cancer Res. 74 (11): 2936-2945). This approach predicts signaling pathway activity based on knowledge-based Bayesian computational models, which interprets quantitative transcriptome data as functional output of an active signaling pathway, by using expression levels of transcriptional target genes. This approach enables a more informed choice of therapy and improved prediction of targeted therapy response.

A comprehensive characterization of tumors cells therefore requires analyzing a tumor cell's transcriptome as well as its genome such that the expression levels permit determining the signaling pathways specifically employed in the cell as well as the analysis of mutations for determining why a specific signaling pathway is utilized in said tumor cell.

For cancer patients suffering from multiple tumors and/or tumors present at difficult to reach places, it may not be possible to obtain biopsies from the tumor(s) or all the tumors. This drawback has led to the clinical development of assessing circulating tumor cells (CTCs). Circulating tumor cells are cells that have been shed from a tumor and entered the blood circulation. CTCs can be obtained from a patient through a simple venipuncture, and analyzing characteristics of single CTCs may be used to monitor tumor characteristics such as hormonal response, inter- or intra-tumor heterogeneity.

Due to the putative presence of multiple tumors, the heterogeneity of tumors and/or the heterogeneity of different cells from an individual tumor, it is necessary to obtain and analyze multiple CTCs from a single patient for providing best possible diagnosis and treatment prescriptions. Moreover, it is essential—when analyzing multiple CTCs from a single patient—that the transcriptome and the genomic profile or genomic characterization (i.e. the presence or absence of particular mutations and/or the presence or absence of particular markers) for each individual CTC of said multiple CTCs can be obtained, analyzed and correlated. This requirement provides that the individual CTCs can be analyzed separately from each other, and that the extra-nuclear constituents and the nuclear constituents of each individual CTC can be obtained separately from each other for the subsequent analysis thereof such that the nuclear and the extra-nuclear constituents, in particular the nuclear and the extra-nuclear nucleic acids, can be subjected to different analytical methods.

Isolation and subsequent characterization of CTCs from a blood sample is technically challenging due to the low numbers among an abundance of white blood cells. Nevertheless, for analyzing and/or characterizing CTCs or other cells, it is desirable to isolate these cells by passive trapping, i.e. without using antibody- or ligand-based capture technologies or application of other external forces such as electric forces, before processing these single cells (e.g. CTCs) for characterizing at least one of their constituents.

Microfluidic devices for trapping, isolating, and processing single cells are described in prior art.

The US 2015/0018226 A1 discloses a microfluidic device for trapping, isolating, and processing single cells. The device includes a cell capture chamber having a cell funnel positioned within the cell capture chamber to direct a passing cell through the capture chamber towards one or more cell traps which are positioned downstream of the funnel to receive a cell. In this device, the cell capture chamber is positioned in-flow direction.

The WO 2003/085379 A2 discloses a system for microfluidic manipulation and/or detection of cells or particles allowing a broad range of assays. The manipulation enables controlled input, movement/positioning, retention/localization, treatment, measurement, release and/or output of the particles. The system comprises sample chambers for receiving multiple cells.

The US 2007/0264705 A1 discloses an apparatus for handling cellular entities, wherein said apparatus comprises a first substrate having an array of first wells open to a first major surface of the first substrate, said first wells being adapted to hold a cellular entity. The apparatus further comprises fluidic channels open to each well such that all of the first wells are in fluid connection with a second well via a common channel which prevents separate analysis of the nuclear and extra-nuclear constituents of each individual cell captured in the plurality of first wells.

The US 2011/0262906 A discloses a sequential flow analysis tool comprising a microfluidic device having a fluid path defined within a substrate between an input and an output. The device includes a capture chamber provided within but offset from the fluid path, the capture chamber extending into the substrate in a direction substantially perpendicular to the fluid path such that operably particles are provided within a fluid flowing within the fluid path will preferentially collect within the capture chamber. The capture chamber has no separate fluidic connection to an auxiliary chamber in which constituents of captured cells can be analyzed.

The US 2012/0053329 A1 discloses a method to prepare DNA, RNA, and protein from one cell type. In an example, peripheral blood is first hemolyzed, and the resulting solution is passed through a filter having a pore size capable of capturing white blood cells. The filter can then be washed to wash off components that become a noise in expression analysis, such as haemoglobin in red blood cells. Then the cells on the filter are reacted with a surfactant, and the surfactant-treated solution is passed through a second filter having a pore size that is smaller than the cell size and larger than the nucleus size. By passing through the filter pores under pressure, cell membranes dissolved by the surfactant are lysed, while nuclear membranes that are undissolved by the surfactant and are relatively robust against physical stimulation such as pressure pass through the filter pores without being lysed. Apparently, the method is suitable for recovering the nuclei from a plurality of cells. However, the method has not been designed for separately recovering the nuclear and extra-nuclear constituents of a single cell.

The WO 2013/130714 A1 discloses systems and devices for multiple single cell capturing and processing utilizing microfluidics. Embodiments of the micro fluidic device are configured to capture single cells at discrete locations (niches). Said niches comprise a small gap such that a cell entering the niche blocks the gap and prevents any further flow into the niche. The niche gap is sufficiently small that cells may be captured at the operational pressure/flow level. A buffer inlet may converge with a cell inlet so as to force cells to a side of a feeder channel that is closest to a series of transverse cell capture channels. The resistance for the transverse cell capture channels may be lower than that of a cell overflow channel to induce preferential flow of cells into niches versus into the cell overflow channel. A fluidic connection or the niche gaps to an auxiliary chamber for processing/analyzing constituents of the captured cells is not disclosed.

The US 2014/0212881 A1 discloses a system and method for capturing and analyzing a set of cells, comprising: an array including a set of parallel pores, each pore including a chamber outlet, and configured to hold a single cell, and a pore channel fluidly connected to each chamber inlet of the set of parallel pores; an output channel fluidly connected to each pore channel of the set of parallel pores; a set of electrophoresis channels fluidly coupled to the output channel, configured to receive a sieving matrix for electrophoretic separation; and a set of electrodes including a first electrode and a second electrode, wherein the set of electrodes is configured to provide an electric field that facilitates electrophoretic analysis of the set of cells. All pores end in a common fluidic channel such that the system does not enable individual analyses of single cells, and it is intended to individually encapsulate the cells within an encapsulating matrix before the captured cells are processed.

The US 2015/0125865 A1 discloses methods and systems for merging a droplet with a volume of fluid in a microfluidic system. The methods use a microfluidic structure designed to merge a fluid with a droplet in order to dilute, add volume, or add selected reagents, biological materials, or synthetic materials to a droplet.

There is a demand for microfluidic devices which allow capturing a single cell, and to differentially analyze different constituents of the cell being captured, in particular nuclear and extra-nuclear nucleic acids of the cell.

### SUMMARY OF THE INVENTION

According to a first aspect, the present invention provides a micro fluidic device comprising a microfluidic structure for differential extraction of nuclear and extra-nuclear constituents of a single cell.

According to a second aspect, the present invention provides a method for manufacturing a microfluidic device comprising a microfluidic structure for differential extraction of nuclear and extra-nuclear constituents of a single cell.

According to a third aspect, the present invention provides the use of a microfluidic device comprising a micro fluidic structure for differential extraction of nuclear and extra-nuclear constituents of a single cell.

According to a further aspect, the present invention concerns a method of differentially extracting nuclear and extra-nuclear constituents of a single cell.

The microfluidic structure of the microfluidic device according to the first aspect comprises a feeding channel, at least one trapping structure for capturing a single cell, and an output channel for receiving constituents of the cell upon lysis of the cell.

The term “cell” as used herein refers to living cells, preferably to eukaryotic cells, more preferably to mammalian cells, and most preferably to human cells.

The feeding channel comprises a first end and a second end. The feeding channel's first end is an open end and represents an inlet for providing cells to be captured to the micro fluidic structure of the micro fluidic device.

In an embodiment, the inlet comprises a fitting for attaching a reservoir—such as a bag or syringe—containing cells to be captured. In another and/or alternative embodiment, the fitting is a female Luer-Lok fitting.

The feeding channel comprises a second end. Said second end is an open end. Said second end of the feeding channel is an outlet. In an embodiment of the feeding channel, the second end of the feeding channel is in fluid communication with a waste reservoir. Said waste reservoir is configured for receiving liquid that is flowing through the feeding channel as well as cells that are not captured by the trapping structure.

In an additional and/or alternative embodiment, the feeding channel has an inner width of at least about 20 μm, preferably of at least about 30 μm, more preferably of at least about 35 μm, and most preferably of about 40 μm. The feeding channel has an inner width of less than about 100 μm, preferably of less than about 60 μm, more preferably of less than about 50 mm. The inner width of the feeding channel is ideally between about 35 μm and about 45 μm.

In an additional and/or alternative embodiment, the feeding channel has a height of ≤50 μm, preferably a height in the range of about 8 μm to about 20 μm, more preferably in the range of about about 10 μm to about 15 μm, most preferably of about 10 μm.

The microfluidic structure further comprises a trapping structure for capturing a cell migrating through the feeding channel. The trapping structure is configured as a bulge of the feeding channel, said bulge extending orthogonally from one side of the flow path within the feeding channel. The axis of the trapping structure extends essentially perpendicular from the longitudinal axis of the feeding channel in the section of the feeding channel where the trapping structure bulges is located. Thus, the trapping structure for capturing a single cell is not positioned within the flow path of the feeding channel.

In additional and/or alternative embodiments, the trapping structure has a rectangular, square, round or oval cross section. In an additional and/or alternative embodiment, the trapping structure is a conical or funnel-shaped bulge of the feeding channel's lumen. In another embodiment, the trapping structure is wedge-shaped.

The trapping structure comprises an open 1^{st} end and an open 2^{nd} end. The open 1^{st} end is in fluid communication with the lumen of the feeding channel, whereas the open 2^{nd} end is in fluid communication with an output channel.

In an additional and/or alternative embodiment, the open 1^{st} end and the open 2^{nd} end of the trapping structure are arranged at opposite ends of the trapping structure.

In an additional and/or alternative embodiment, the open 1^{st} end of the trapping structure has a cross-sectional diameter such that typically only a single cell is captured in an individual trapping structure of the trapping device. The cross-sectional diameter of the aperture at the open 1^{st} end preferably is not larger than two times the size of the cell to be captured. Preferably, the aperture of the open 1^{st} end of the trapping structure has a width or cross-sectional diameter in the range of between about 8 μm to about 20 μm, e.g. 8 μm, 9 μm, 10 μm, 11 μm, 12 μm, 13 μm, 14 μm, 15 μm, 16 μm, 17 μm, 18 μm, 19 μm or 20 μm.

The aperture of the open 2^{nd} end of the trapping structure has a width or cross-sectional diameter in the range of between about 1 μm and about 5 μm, e.g. 1 μm, 2 μm, 3 μm, 4 μm or 5 μm, preferably about 4.5 μm.

In an alternative and/or additional embodiment, the width or cross-sectional diameter of the open 2^{nd} end's aperture is smaller than the width or cross-sectional diameter of the open 1^{st} end's aperture.

In an alternative and/or additional embodiment, the angle α between the wall of the trapping structure (representing the hypotenuse) and a perpendicular dropped from the outer edge of the aperture of the open 1^{st} end (the adjacent cathetus) is in the range of between about 3° to about 10°, e.g. 3°, 4°, 5°, 6°, 7°, 8°, 9° or 10°.

The length of the open 1^{st} end and/or the open 2^{nd} end of the trapping structure may be the same as the width of the same open end. In an alternative embodiment, the length of the open 1^{st} end and/or the open 2^{nd} end differs from the width of the same open end. For example, due to manufacturing of the microfluidic device, the length of the open 1^{st} end and/or the open 2^{nd} end of the trapping structure is about 10 μm.

In an additional and/or alternative embodiment, the width and height of the aperture of the open second end of a wedge-shaped trapping structure is 4 μm by 10 μm or 4.5 μm by 10 μm.

The trapping structure is in fluid communication with an output channel. The output channel comprises a first end and a second end. The output channel's first end in an open end having an aperture. Said aperture is in fluid connection with the aperture of the trapping structure's second open end. Said fluid connection provides a narrow section, the inner diameter or width of which being such that a cell captured in the trapping device can not pass through said fluid connection at operable pressure/flow rates. More specifically, the dimension of the inner diameter or width of the fluid connection is such that the nucleus of a captured cell can not pass through at operable pressure/flow rates too. Preferably, the narrow section/fluid connection has an inner diameter or width in the range of about 1 μm to about 4 μm.

The second end of the output channel is an open end. In a preferred embodiment, said second end of the output channel fluidly connectable with at least one auxiliary chamber. Preferably, said at least one auxiliary chamber is a reaction chamber for analyzing and/or amplifying constituents obtained from the cell caught in the trap.

In an additional and/or alternative embodiment, the output channel has an inner diameter in the range of between about 25 μm and about 35 μm. A preferred embodiment of an output channel has a width of between about 25 μm and about 35 μm, and a height of about 10 μm.

In an additional and/or alternative embodiment, the output channel is branched. That it, the output channel comprises two or more second ends. Hence, the output channel of this embodiment comprises two or more legs. Preferably, each leg provides a flow path to a separate auxiliary chamber.

In an additional embodiment, the two or more legs of the output channel are provided with one or more valves. Said valves allow to determine which flow path is used at any time, and permits changing the flow path through the output channel along one or another leg of the output channel. This embodiment is advantageous for directing constituents obtained from the cell to separate/different auxiliary chambers for separate further processing and/or analysis. For example, the auxiliary chambers may be configured for performing nucleic acid amplification reactions such as polymerase chain reactions.

In an additional and/or alternative embodiment, the microfluidic device comprises a plurality of trapping structures, wherein each trapping structure of said plurality of trapping structures is in fluid communication with a separate output channel. This configuration is advantageous in that the extra-nuclear constituents and the nuclear constituents of each individual cell being trapped in the microfluidic device can be individually and separately transferred into individual compartments for subsequent individual analyses. Such a configuration is essential for CTC analyses, because it enables separate analysis of the extra-nuclear constituents and the nuclear constituents of individual CTCs, for example for determining the individual CTCs transcriptomes and genetic profiles.

In an additional and/or alternative embodiment, the microfluidic structure further comprises at least one buffer channel for supplying one or more buffers to the feeding channel. In an preferred embodiment, the micro fluidic structure comprises two buffer channels. The at least one buffer channel is configured for guiding the cells flowing in the feeding channel towards the side of the feeding channel where the trapping structure is located and/or for supplying a lysis buffer to the captured cell.

Said at least one buffer channel has a 1^{st} end and a 2^{nd} end. The 1^{st} end of the at least one buffer channel is an open end. In an embodiment, the 1^{st} end of the at least one buffer channel is in fluid communication with a buffer reservoir for supplying buffer to the buffer channel within the microfluidic structure. In an additional and/or alternative embodiment, the 1^{st} end of the at least one buffer channel comprises a fitting for attaching a reservoir such as, for example, a bag or. In an embodiment, the fitting is a Luer-Lock fitting, preferably a female Luer-Lock fitting.

In embodiments of the microfluidic structure having two or more buffer channels, it is preferred that each of the two or more buffer channels is in fluid communication with a separate buffer reservoir.

The 2^{nd} end of the at least one buffer channel is an open end. Said open end is an aperture that is in fluid communication with the feeding channel. Said aperture is an outlet for providing the buffer flowing within the buffer channel to the feeding channel. The outlet of the at least one buffer channel is positioned at the opposite side of the at least one trapping structure with respect to the feeding channel's cross section. The outlet of the at least one buffer channel is not positioned directly opposite the trapping structure, but at a distance before the trapping structure, with respect to the direction of flow through the feeding channel.

In an additional and or alternative embodiment, the at least one buffer channel is fluidly connected to the feeding channel in a tilted orientation such that the flow path within the at least one buffer channel converges with the flow path of the feeding channel in a sharp angel, i.e. in an angle of smaller than 90°. The angle between the feeding channel and the at least one buffer channel is in the range of about 30° to about 70°, preferably in the range of about 40° to about 60°, and most preferably in the range of about 45° to about 55°. In an additional and/or alternative embodiment, the angle is about 50°. The tilting of the at least one buffer chamber is advantageous in that the flow of buffer from the at least one buffer chamber drive migration of the cells along the feeding channel from the cell inlet towards the waste outlet. In addition, the flow of buffer from the at least one buffer channel forces the cell migrating along the feeding channel towards the side of the feeding channel bearing the trapping structure by. This configuration increases the efficacy of capturing a cell flowing through the feeding channel by the trapping structure.

In additional and/or alternative embodiments, the microfluidic structure comprises one or more valves for opening and/or closing specific flow paths in the microfluidic structure, and for directing the flow of liquid through the channels of the microfluidic structure and microfluidic device. For example, the cell inlet and/or the waste outlet of the feeding channel may be provided with a valve, the buffer inlet and/or the outlet of each buffer channel may be provided with a valve, and/or the inlet and/or outlet of the output channel may be provided with a valve.

In an additional and/or alternative embodiment, one or more of the channels of the microfluidic device, i.e. the buffer channel(s), the feeding channel and/or the output channel (including any one of its legs), comprises walls, wherein the angle α between the wall of the channel (representing the hypotenuse) and a perpendicular dropped from the outer edge of the channel's bottom (the adjacent cathetus) is in the range of between about 3° to about 10°, e.g. 3°, 4°, 5°, 6°, 7°, 8°, 9° or 10°. Preferably, all channels of the microfluidic device possess walls wherein the angle α between the wall of the channel (representing the hypotenuse) and a perpendicular dropped from the outer edge of the channel's bottom (the adjacent cathetus) is in the range of between about 3° to about 10°, e.g. 3°, 4°, 5°, 6°, 7°, 8°, 9° or 10°. Such configuration of the channels is advantageous during its manufacturing, because the mold for injection molding the microfluidic device can be removed from the microfluidic device after being injection molded more easily and with less risk of damaging microfluidic structures within the microfluidic device.

In an additional and/or alternative embodiment, the microfluidic device comprises at least one auxiliary chamber for further processing nuclear and/or extranuclear constituents of a single cell. The term “processing” in this regard comprises reaction for analyzing, detecting, characterizing, amplifying and/or sequencing a constituent of a cell. The at least one auxiliary chamber may be integral part of the microfluidic structure such that the at least one auxiliary chamber is in fluid connection with the output channel. In an alternative embodiment, the at least one auxiliary chamber is connectable to the output channel for establishing a fluid connection for transferring the cell's constituents into the auxiliary chamber. The latter embodiment has the advantage that different auxiliary chambers can be connected to the output channel for differently processing nuclear and extra-nuclear constituents of the cell.

A micro fluidic device comprising a micro fluidic structure according to the invention is advantageous in that the cells to be captures can be captured while in a fluid (such as a FACS flow or PBS (=phosphate buffered saline) buffer) maintaining integrity of the cell, and that the cell can subsequently be lysed directly in the trapping structure by supplying a lysis buffer such that any of the cell's constituents can be released directly into the output channel which may contain a liquid suitable for further processing the constituents for detection and/or analysis. It has surprisingly been found that additional changes of fluids and washing steps, which are typically used when cells are lysed, are not necessary. The amount of lysis buffer transferred into the output channel is neglectable with respect to its effect on inhibiting subsequent reactions for determining and/or analyzing a cell's constituent. For example, a typical lysis buffer for isolating DNA contains salts and a surfactant which are known to inhibit amplification of DNA fragments by polymerase chain reaction.

Without wishing to be bound, it is believed that the advantage of the microfluidic structure is based on the configuration wherein the main flow direction in the feeding channel is orthogonal to the fluidic direction in the trapping structure towards the outlet and the ratio between the cross-sectional area of the feeding channel and the cross-sectional area of the outlet aperture of the trapping structure/narrow section. It is believed that these features contribute to the fact that only a minute, neglectable amount of lysis buffer accesses the output channel upon lysis of a cell captured in the trapping structure. For example, the volume of a trapping structure measuring ½×15 μm×15 μm×10 μm is equivalent to about 1.1 pl. Thus, if the entire content of such a trap enters the output channel bearing a volume of about 10 μl of prefilled fluid, the amount of lysis buffer sums up to only 1:10⁷. This minor amount of lysis buffer in the buffer within the output channel does not affect subsequent analysis and/or amplification of specific cell constituents. Even if a lysis buffer containing guanidinium (CH₅N₃) salts is used, the residual amount thereof does not affect subsequent nucleic acid amplification, even if the amplification reaction is performed in said 10 μl volume.

Guanidinium salts can be used in the rapid purification of nucleic acids directly from serum or urine. However, a silica membrane or silica coated beads have to be used to collect/bind the nucleic acids to those beads/membranes, before washing away the guanidinium salts by isolating the beads or washing the membranes. In using the microfluidic device described herein, neither a silica membrane nor silica coated beads are required, not even an extra step for washing away the guanidinium salt when the cell's constituent to be isolated is a nucleic acid.

In an additional and or alternative embodiment, the microfluidic structure is configured such that the ratio of the volume of the trapping structure to the volume of the output channel is at least about 1:10³, at least about 1:10⁴, at least about 1:10⁵, at least about 1:10⁶ or even at least 1:10⁷.

In additional and/or alternative embodiments, the microfluidic device comprises one or more of said microfluidic structures. In additional and/or alternative embodiments, the microfluidic device comprises additional microfluidic structures such as, for example, microfluidic structures for pinched flow fractionation of cells, or for performing analyzing and/or amplifying reactions using the constituents obtained from captured cells.

The microfluidic device enables differential analysis of the different nucleic acid species of a single cell.

According to the second aspect, the present invention provides a method for manufacturing a microfluidic device comprising a microfluidic structure according to the first aspect. In an embodiment, the microfluidic device is manufactured as a polymeric one-layer device by injection molding. The microfluidic structure is produced by injection molding a suitable polymer, and subsequent sealing the channels with a polymer film, for example by means of UV-assisted thermal bonding of the polymer film to the injection molded structure bearing the microfluidic channels. This manufacturing method permits generating channels having a predefined width and typically the same height. This method of manufacturing microfluidic structures as such in known in the technical field of microfluidic devices.

According to the third aspect, the invention provides the use of a microfluidic structure according to the first aspect for differentially extracting nuclear and extra-nuclear constituents of a single cell. The use comprises capturing a single cell in the at least one trapping structure of the microfluidic structure, lysing the cell while maintaining integrity of the cell's nucleus, and subsequently lying the cell's nucleus such that extra-nuclear and the nuclear constituents of the cell are released successively to be processed separately from each other. In an additional embodiment, the use of the microfluidic structure according to the first aspect comprises subsequent analyzing/characterizing at least one of the nuclear and/or extra-nuclear constituents of the cell.

In an additional and/or alternative embodiment, the nuclear constituents of the cell and/or the extra-nuclear constituents of the cell are nucleic acid molecules. The nuclear nucleic acid is preferably the cell's DNA. The extra-nuclear nucleic acid is preferably the cell's mRNA.

In using the the microfluidic device for differentially extracting nuclear and extra-nuclear constituents of a single cell the method described herein after can be employed.

Thus, in yet another aspect the present invention provides a method for differentially extracting nuclear and extra-nuclear constituents of a single cell.

The use/and or the method comprises the steps of:
providing at least one cell to the feeding channel of a microfluidic device as described herein before;
capturing the at least one cell in the at least one trapping structure;
lysing the cell captured in the at least one trapping structure without affecting integrity of the cell's nucleus by supplying a first lysis buffer to the cell;
releasing the extra-nuclear constituents of the cell into the output channel;
transferring the extra-nuclear constituents of the cell from the output channel into an auxiliary chamber for further processing;
lysing the cell's nucleus by supplying another lysis buffer to the nucleus;
releasing the constituents of the cell's nucleus into the output channel; and
transferring the constituents of the cell's nucleus from the output channel to an auxiliary chamber for further processing.

For providing at least one cell to the feeding channel, one or more cell are present in a fluid which maintains integrity and viability of the cells. Said fluid is an isotonic fluid, for example a FACSflow-buffer or PBS. Said fluid containing the at least one cell is provided to the cell inlet of the feeding channel such that the fluid enters the feeding channel at its cell inlet. Optionally a force may be exerted for securing that the fluid is flowing through the feeding channel at a desired flow rate. Preferably, the flow rate of the fluid is in the range of between 2.9 μL/h to about 5.7 μL/h. This may—depending on the dimensions of the channels—correspond to a pressure in the range between about 2 mbar to about 10 mbar, preferably from about 3 mbar to about 5 mbar.

A cell being present in said fluid enters the feeding channel at its first end and migrates along the feeding channel due to the flow of fluid until the cell passes the trapping structure. The cell enters the trapping structure due to the microfluidic dynamics within the microfluidic structure, and is captured in the trapping structure. The cell being captured in the trapping structure clogs the aperture at the trapping structure's 2^{nd} end.

In a preferred embodiment, additional fluid maintaining integrity and viability of the cell is supplied to the feeding channel from at least one separate buffer reservoir via the buffer channel or via at least one of the buffer channels. The converging flows of fluids in the feeding channel drives migration of the cells along the flow path of the feeding channel, and towards the side of the feeding channel opposite to the outlet of the buffer channel supplying the buffer or medium. A single cell is then captured in the at least one trapping structure present along the subsequent flow path within the feeding channel when the cell passes the position of one of the trapping structures. As long as a cell is captured in a trapping structure no further cell can be trapped in the same trapping structure.

The use and/or method further comprises lysing the cell being captured in the trapping structure. The cell is lysed such that the integrity of the cell's nucleus is not affected. For lysing the cell, a first lysis buffer is supplied to the feeding channel and to the captured cell after cells which are not captured in a trapping structure of the microfluidic device are removed from the feeding channel. Supplying the first lysis buffer to the feeding channel may be performed using the cell inlet of the feeding channel. In an additional and/or alternative embodiment, the first lysis buffer is supplied to the feeding channel and to the captured cell via the buffer channel or via at least one of the buffer channels. Hence, the same buffer channel supplying the fluid maintaining integrity and viability of the cell or another buffer channel may be used for supplying the first lysis buffer to the feeding channel/trapping structure/captured cell. Supplying the first lysis buffer via at least one of the buffer channels is advantageous in that once a cell, or a number of cells being captured when multiple trapping structures are present along the feeding channel, the process of lysing the cells can immediately be started.

In an additional embodiment, the first lysis buffer consists of 0.5×TBE containing 0.5% (v/v) Triton X-100. Thus, this first lysis buffer consists of an aqueous solution containing 44.5 mM Tris-Borate, 1 mM EDTA and 0.5% (v/v) Triton X-100. The first lysis buffer does not affect integrity of the cell's nucleus, but leaves it intact. This first lysis buffer is particularly suitable for analysing the cell's transcriptome by subsequent reverse transcription and PCR amplification of mRNA molecules of the cell.

The extra-nuclear constituents of the captured cell are then release from the trapping structure into the narrow section of the output channel connecting the outlet at the 2^{nd} end of the trapping structure with the inlet of the output channel, wherein no or only a neglectable minute amount of the first lysis buffer enters said narrow section.

In an alternative and/or additional embodiment, the output channel contains a buffer or fluid suitable for performing the desired reaction(s) for analysing and/or characterizing a extracellular constituent of the cell. Hence, the captured cell is lysed and its constituents are release and transferred to the output channel containing a buffer or fluid, e.g. FACS-flow, PBS, a PCR buffer or nuclease-free water, that does not hamper subsequent detection and/or more specific molecule(s) of the cell, such as a specific protein, a nucleic acid sequence and/or a metabolite.

In a further step, the cell's extra-nuclear constituents are transferred from the narrow section to the output channel and are transferred from the output channel to an auxiliary chamber for further processing, i.e. for detection and/or analysis.

Upon lysing the cell with said first lysis buffer, integrity of the cell's nucleus is not affected. Due to the dimensions of the trapping structures outlet and the narrow section, the intact nucleus can not pass through the outlet at the trapping structure's 2^{nd} end and the narrow section into the output channel, but is captured in the trapping structure.

In a further step, the nucleus of the cell is lysed. The nucleus is lysed in that a second lysis buffer, the composition of which is different from the composition of the first lysis buffer, is supplied to the feeding channel and to the nucleus being captured in the trapping structure. Supplying the second lysis buffer to the feeding channel may be performed using the cell inlet of the feeding channel. In an additional and/or alternative embodiment, the second lysis buffer is supplied to the feeding channel and to the captured nucleus via the buffer channel or via at least one of the buffer channels. Hence, the same buffer channel supplying the first lysis buffer and/or another buffer channel may be used for supplying the second lysis buffer to the feeding channel/trapping structure/captured cell. Supplying the second lysis buffer via another buffer channel that the first lysis buffer is particularly advantageous in that, the process of lysing the nucleus can immediately be started.

In an additional embodiment, the second lysis buffer consists of 0.5×TBE containing 0.5% (v/v) Triton X-100 supplemented with protease K, preferably with a 1:70 dilution of a protease K. Thus, this second lysis buffer consists of an aqueous solution containing 44.5 mM Tris-Borate, 1 mM EDTA, 0.5% (v/v) Triton X-100 and protease K.

The nuclear constituents of the cell are then release from the trapping structure into the narrow section of the output channel connecting the outlet at the 2^{nd} end of the trapping structure with the inlet of the output channel, wherein no or only a neglectable minute amount of the second lysis buffer enters said narrow section.

In an alternative and/or additional embodiment, the output channel contains a buffer or fluid suitable for performing the desired reaction(s) for analysing and/or characterizing a extracellular constituent of the cell. Hence, the nucleus is lysed and its constituents are release and transferred to the output channel containing a buffer or fluid, e.g. FACS-flow, PBS, a PCR buffer or nuclease-free water, that does not hamper subsequent detection and/or more specific molecule(s) of the cell, such as a specific protein, a nucleic acid sequence and/or a metabolite.

In a further step, the cell's nuclear constituents are transferred from the narrow section to the output channel and are transferred from the output channel to an auxiliary chamber for further processing, i.e. for detection and/or analysis. Preferably, the auxiliary chamber is onother, optionally different, auxiliary chamber than the auxiliary chamber the extra-nuclear constituents were transferred to.

In additional embodiment, the method further comprises:
amplification of at least one nucleic acid sequence of the cell's nuclear constituents; and
amplification of at least one nucleic acid sequence of the cell's extra-nuclear constituents.

In an additional embodiment, the method further comprises analyzing the nucleotide sequence of the amplification product of the at least one nucleic acid sequence of the cell's nuclear constituents.

The method does not require separate washing steps after the cell and/or the nucleus have been lysed for removing residual lysis buffer containing compound affecting or even impairing a subsequent analysis of constituents. This reduces time and chemicals required for analyzing cells, and thus costs. Thus, in alternative and/or additional embodiments, the method is performed without one or more washing steps after lysing the cell and/or without one or more washing steps after lysing the nucleus.

In addition, the method permits a much more accurate and reliable way of analysing single cell, in particular for correlating genomic information with transcriptome information. Since washing steps are not required, the likelihood of recovering all DNA molecules of a single cell increases significantly, because each additional step in the process of isolating DNA, especially washing steps, bear the risk of removing DNA from the sample. For instance, for single cell DNA/genomic analysis this is problematic as DNA molecules of the cell being washed away cannot be recovered. This is particularly relevant for DNA/genomic analysis of single cells, because as each cell has only 2 copies of each of its chromosomes. Furthermore, washing steps might influence the RNA profile. However, the original RNA profile of the cell, i.e. the RNA profile of the cell in its natural environment, is required for an accurate transcriptome and pathway analysis. The method according to the invention provides a method wherein the RNA profile of a cell given in the cells neutral environment is least affected. Therefore, the method provides a more accurate and reliable analysis/characterization of single cells might become impossible.

Therefore, the method according to the invention has the crucial advantage that the DNA is obtained separately from the RNA from the same cell, and that the abundance of both types of nucleic acids is not significantly affected.

Polymerase chain reactions revealed the this method indeed enables isolating and amplifying both the RNA and the DNA from the same single cell. In an experiment, a single cell was captured and its extra-nuclear nucleic acids, and its nuclear nucleic acids were subsequently and separately recovered as described herein before. Quantitative comparison of an amplification product of a fragment of the beta-actin mRNA isolated form a single cell and being reverse transcribed using a oligo-dT-primer reveled an amount of mRNA equivalent to 3.5 cells (FIG. 3A). A fragment of the RNase P DNA isolated from the same single cell using specific primer could have been amplified too (FIG. 3B). Compared with the amplification of a positive control containing 2 ng genomic DNA of the same cell type, it took 10 more amplification cycles to obtain the same amount of amplification product.

These data prove that mRNA and genomic DNA were isolated separately from the same single cell. Therefore, the method allows executing a complete pathway analysis of a single cell comprising an RNA analysis to assess which pathway is involved, and a DNA analysis to assess where and why a pathway is deregulated.

### BRIEF DESCRIPTION OF THE DRAWINGS

These and other aspects of the invention will be apparent from and elucidated with reference to the embodiments described hereinafter. Such embodiment does not necessarily represent the full scope of the invention, however, and reference is made therefore to the claims and herein for interpreting the scope of the invention.

In the drawings:

FIG. 1 shows a schematic illustration of an embodiment of a micro fluidic structure in accordance with the invention.

FIG. 2 shows a schematic illustration of another embodiment of a microfluidic structure in accordance with the invention.

FIG. 3A and FIG. 3B display graphs illustrating the amplification of nucleic acid sequenced of a single cell isolated by a method according to the invention.

FIG. 4 displays a cross sectional view of a channel in a preferred embodiment of the microfluidic device.

### DETAILED DESCRIPTION OF EMBODIMENTS

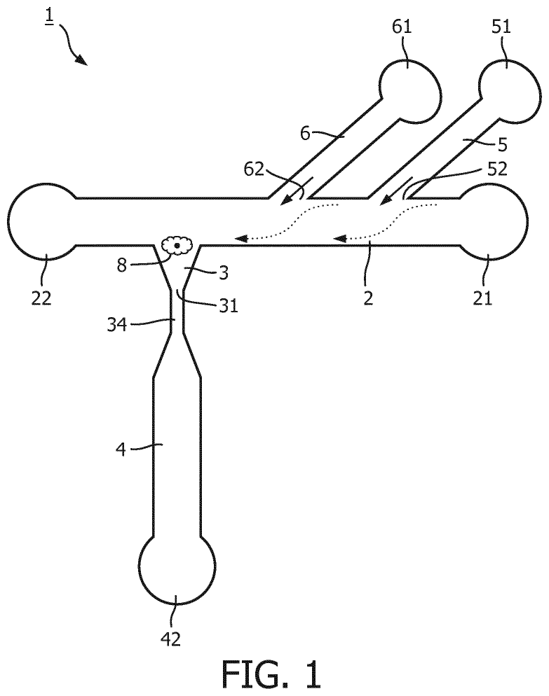

Referring to FIG. 1, a schematic illustration of an embodiment of a microfluidic structure in accordance with the invention is shown. The microfluidic structure 1 comprises a feeding channel 2 possessing an inlet (cell inlet) 21 and a waste outlet (22). The microfluidic structure 1 comprises a trapping structure 3 in fluid communication with and orthogonally extending from the flow path of the feeding channel 2. The trapping structure 3 comprises an outlet 31 in fluid connection with an output channel 4. The fluid connection 34 between the trapping structure 3 and the output channel 4 provides a narrow section configured to prevent a cell 8 being captured in the trapping structure 3 from accessing the output channel 4. The output channel 4 possesses an outlet 42 which is or may get fluid connection with an auxiliary chamber which is configured for detecting and/or analyzing one or more cell constituents. The microfluidic structure 1 further comprises two buffer channels, a first buffer channel 5 and a second buffer channel 6. The first buffer channel 5 being in fluid communication with a first buffer reservoir 51, and the second buffer channel 6 in fluid communication with a second buffer reservoir 61. Optionally one of the first buffer reservoir 51 and the second buffer reservoir 61 contains a fluid maintaining integrity and viability of cells, whereas the other buffer reservoir contains a lysis buffer for lysing a cell captured in the trapping structure 3.

During operation, a flow of buffer or medium is provided via at least one of the buffer channels 5, 6 as indicated by the solid arrows. A cell migrating along the feeding channel 2 is forced within the feeding channel 2 towards the side opposite of the outlet 62 of the buffer channel 5 and/or 6 to be captured by the trapping structure 3 also located at the side of the feeding channel opposite to the outlets 52, 62 of the buffer channels 5, 6.

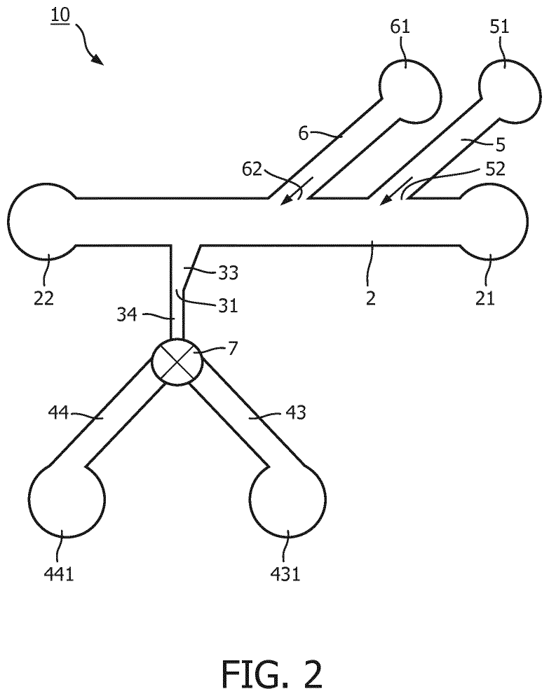

Referring to FIG. 2, another embodiment of the microfluidic structure according to the invention is schematically shown. The trapping device 33 has a wedge-shaped form provided that the trapping structure has a rectangular or square cross section. The microfluidic structure 10 comprises an output channel having a first leg 43 and a second leg 44, wherein the first leg 43 possesses an outlet 431 which is or may become in fluid communication with a first auxiliary chamber, and wherein the second leg 44 possesses an outlet 441 which is or may become in fluid communication with a second auxiliary chamber.

The output channel of the embodiment shown in FIG. 2 further comprises an actuatable two-way valve 7 for directing the flow coming from the trapping structure to one of the two legs 43, 44 of the output channel.

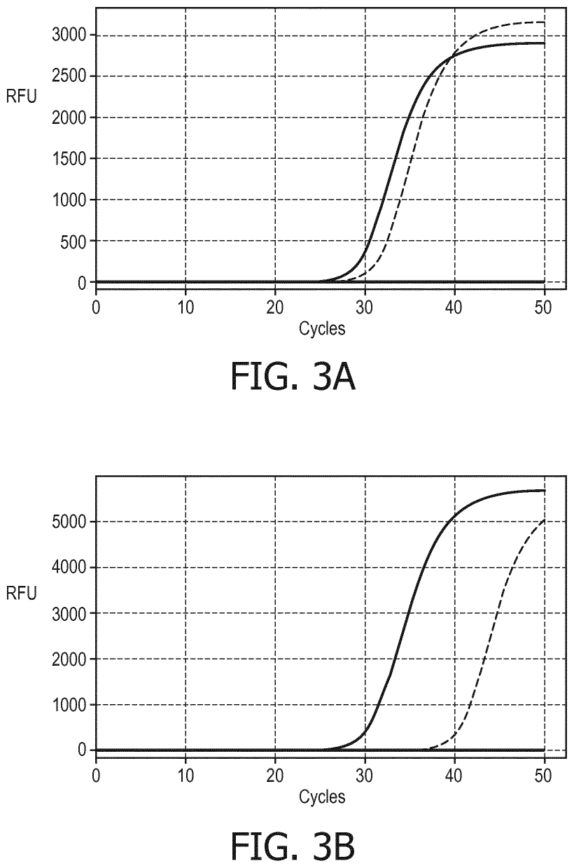

Referring to FIG. 3A a graph is shows visualizing the results of amplifications of a fragment of β-actin mRNA of a single cell captured by using a microfluidic device according to the invention. The mRNA of the cell was obtained by the method according to the present invention. The fragment was amplified in a real-time PCR using specific primers after reverse transcription of the cell's mRNA using an oligo-dT-Primer. Increase of fluorescence upon the amplification cycles were monitored for the cell's mRNA (dashed line) and from an amount of mRNA equivalent to 3.5 cells (solid line) as positive control. A negative control without mRNA did not lead to any detectable fluorescence.

Referring to FIG. 3B a graph is shown which visualizes the results of amplifications of RNAse P DNA. The genomic DNA was obtained from the same single cell as the mRNA used in the amplification reaction shown in FIG. 3A. The genomic DNA was first subjected to whole genome amplification (WGA). The product of the WGA was diluted to enable real-time PCR amplification of a fragment of the RNase P gene. Increase of fluorescence upon the amplification cycles were monitored for the genomic DNA of the single cell (dashed line) and for 2 ng genomic DNA as positive control (solid line). A negative control without DNA did not lead to any detectable fluorescence.

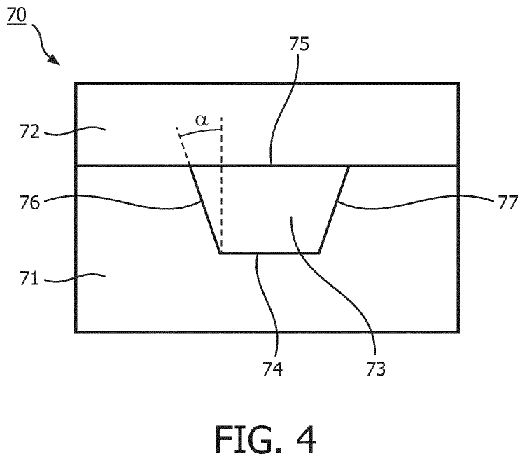

FIG. 4 illustrates a preferred configuration of the channels within an embodiment of the microfluidic device. A cross sectional view of a region of a microfluidic structure 70 comprising a channel 73 is schematically presented. The microfluidic structure 70 comprises a base 71, i.e. a polymeric one-layer device comprising the channel 73, and a lid 72 for sealing the channel 73. The lid 72 may be a polymer film. The channel 73 may be any channel of the micro fluidic structure such as the feeding channel, the buffer channel(s) and/or the output channel (including any legs thereof). The channel 73 is delimitated by its bottom 74, its ceiling 75 and its side walls 76, 77. The angle “α” denotes the angle by which the slope of the side wall deviates from the perpendicular plane with respect to the plane of the bottom of the channel. The angle “α” between wall 76 (representing the hypotenuse) of channel 73 and a perpendicular dropped from the outer edge of the channel's bottom plane (the adjacent cathetus) may in the range of between about 3° to about 10°.

REFERENCE SYMBOL LIST

| | |
|---|---|
| 1 | microfluidic structure |
| 2 | feeding channel |
| 3 | trapping structure |
| 4 | output channel |
| 5 | first buffer channel |
| 6 | second buffer channel |
| 7 | valve |
| 8 | cell |
| 10 | microfluidic structure |
| 21 | cell inlet |
| 22 | waste outlet |
| 31 | outlet |
| 33 | trapping structure |
| 34 | narrow section |
| 42 | outlet |
| 43 | leg |
| 44 | leg |
| 51 | buffer reservoir |
| 52 | outlet |
| 61 | buffer reservoir |
| 62 | outlet |
| 70 | microfluidic structure |
| 71 | base |
| 72 | lid |
| 73 | channel |
| 74 | bottom |
| 75 | ceiling |
| 76 | side wall |
| 77 | side wall |
| 431 | outlet |
| 441 | outlet |

1. A microfluidic device comprising at least one microfluidic structure for differential extraction of nuclear and extra-nuclear constituents of a single cell, said microfluidic structure comprising:
a feeding channel for receiving a volume of a sample containing at least one cell,
at least one trapping structure for capturing a single cell, and
at least one outlet channel in fluid connection with the at least one trapping structure,
wherein the at least one trapping structure extends from one side of the feeding channel substantially perpendicular to longitudinal axis of the feeding channel,
the at least one trapping structure possessing an aperture at its end opposite to the fluid channel and in fluid communication with an outlet channel, said aperture being configured to provide a narrow section such that the nucleus of a cell captured in the trapping structure cannot pass through said narrow section into the outlet channel.

2. The microfluidic device according to claim 1, further comprising at least one buffer channel in fluid connection with the feeding channel, wherein the at least one buffer channel converges with the feeding channel at the side of the feeding channel opposite to the at least one trapping structure, and—with respect to the direction of flow within the feeding channel—at a position along the feeding channel preceding the position of the at least one trapping structure.

3. The microfluidic device according to claim 1, comprising two or more buffer channels.

4. The microfluidic device according to claim 1, wherein the at least one buffer channel or the two or more buffer channel converge(s) with the feeding channel in an angle of less than 90°, preferably in an angle in the range of about 30° to about 70°, more preferably in an angle in the range of about 40° to about 60°, and most preferably in an angle in the range of about 45° to about 55°.

5. The microfluidic device according to claim 1, wherein the narrow section has in inner diameter in the range of about 1 μm to about 4 μm.

6. The microfluidic device according to claim 1, wherein the outlet channel comprises two or more legs.

7. The microfluidic device according to claim 1, wherein the outlet channel or the legs of the outlet channel is/are is in fluid connection with at least one auxiliary chamber for detecting and/or analyzing at least one constituent of the cell.

8. The microfluidic device according to claim 1, wherein the microfluidic structure comprises at least one valve for directing the flow of fluid within the microfluidic structure.

9. The microfluidic device according to claim 8, wherein the inlet and/or the outlet of the feeding channel, the inlet and/or outlet of the at least one buffer channel, the inlet and/or outlet(s) of the outlet channel and/or the diversion within the outlet channel to the legs comprise the valve.

10. A method of manufacturing a microfluidic device as defined in claim 9, wherein the microfluidic structure is produced by injection molding a polymer, and subsequently sealing the channels by bonding a polymer film to the molded structure.

11. Use of a microfluidic device according to claim 9 for differentially extracting nuclear and extra-nuclear constituents of a cell.

12. The use according to claim 11, wherein the nuclear and/or extra-nuclear constituents are nucleic acid molecules.

13. A method for differentially extracting nuclear and extra-nuclear constituents of a single cell, the method comprising the steps of:
providing at least one cell to the feeding channel of a microfluidic device according to claim 9;
capturing the at least one cell in the at least one trapping structure;
lysing the cell captured in the at least one trapping structure without affecting integrity of the cell's nucleus by supplying a first lysis buffer to the cell;
releasing the extra-nuclear constituents of the cell into the outlet channel;
transferring the extra-nuclear constituents of the cell from the outlet channel into an auxiliary chamber for further processing;
lysing the cell's nucleus by supplying a second lysis buffer to the nucleus;
releasing the constituents of the cell's nucleus into the outlet channel; and
transferring the constituents of the cell's nucleus from the outlet channel to an auxiliary chamber for further processing.

14. The method according to claim 13, further comprising:
amplification of at least one nucleic acid sequence of the cell's nuclear constituents; and
amplification of at least one nucleic acid sequence of the cell's extra-nuclear constituents.

15. The method according to claim 14, further comprises analyzing the nucleotide sequence of the amplification product of the at least one nucleic acid sequence of the cell's nuclear constituents.
